SEQUENCE LISTING (2 sequences)

SEQ ID NO: 1 - DNA, 24 bp, Artificial sequence
  Synthetic probe
gatccttctgggaattcctagatc

SEQ ID NO: 2 - DNA, 24 bp, Artificial sequence
  Synthetic probe
gatccttctgggccgtcctagatc

(19) United States
(12) Patent Application Publication
(10) Pub. No.: US 2003/0175270 A1
(43) Pub. Date: Sep. 18, 2003

(54) Methods of regulating cytokine receptor signaling

(76) Inventor: Josef Penninger, Toronto (CA)

Correspondence Address: MARSHALL, GERSTEIN & BORUN, 6300 SEARS TOWER, 233 SOUTH WACKER, CHICAGO, IL, 60606-6357, US

(21) Appl. No.: 10/347,051

(22) Filed: Jan. 17, 2003

Related U.S. Application Data
(60) Provisional application No. 60/349,861, filed on Jan. 17, 2002.

(51) Int. Cl. A61K 39/395

(52) U.S. Cl. 424/141.1; 424/144.1

(57) ABSTRACT

The present invention is related generally to the methods and materials for altering cytokine receptor signaling through the manipulation of the activity of the transmembrane PTPase, CD45.

### CROSS REFERENCE TO RELATED APPLICATION

This application is related to, and claims the priority benefit of, U.S. Provisional Patent Application No. 60/349,861, filed Jan. 17, 2002.

### BACKGROUND OF THE INVENTION

1. Field of the Invention

The present invention relates generally to methods and materials for altering cytokine receptor signaling through the manipulation of the transmembrane PTPase CD45. More specifically, the present invention relates to methods of activating or inhibiting the JAK-STAT signaling pathway (and thus effecting an underlying disease state) via manipulation of CD45 activity.

2. Related Technology

Identified as the first and prototypic transmembrane protein tyrosine phosphatase (PTPase), CD45 has been extensively studied for over two decades and is thought to be important for positively regulating antigen-receptor signaling via the dephosphorylation of Src kinases. However, new evidence indicates that CD45 can function as a Janus kinase (JAK) PTPase that negatively controls cytokine-receptor signaling. A point mutation in CD45, which appears to affect CD45 dimerization, and a genetic polymorphism that affects alternative CD45 splicing are implicated in autoimmunity in mice and multiple sclerosis in humans. CD45 is expressed in multiple isoforms and the modulation of specific CD45 splice variants with antibodies can prevent transplant rejections. In addition, loss of CD45 can affect microglia activation in a mouse model for Alzheimer's disease. Thus, CD45 is moving rapidly back into the spotlight as a drug target and central regulator involved in differentiation of multiple hematopoietic cell lineages, autoimmunity and antiviral immunity.

### SUMMARY OF THE INVENTION

In all of its aspects, the invention is directed to methods of manipulation of the CD45 regulation of the JAK/STAT pathway.

The present invention is directed to methods of activating CD45 phosphatase activity via administration of an anti-CD45 antibody capable of activating CD45 phosphatase activity.

In another of its aspects, the present invention is directed to methods of inhibiting JAKs via administration of an anti-CD45 antibody capable of activating CD45 phosphatase activity.

The present invention is also directed to methods of controlling proliferation of hematopoietic cell lineages via administration of an anti-CD45 antibody capable of activating CD45 phosphatase activity as well as methods of controlling, decreasing, or preventing an inflammation process or disease via the administration of an anti-CD45 antibody capable of activating CD45 phosphatase activity.

In another of its aspects, the present invention is directed methods inhibiting CD45 phosphatase activity via administration of an anti-CD45 antibody capable of inhibiting CD45 phosphatase activity as well as methods of activation of JAKs via administration of an anti-CD45 antibody capable of inhibiting CD45 phosphatase activity.

The present invention is also directed to methods of increasing an immune response by suppressing CD45 activity via administration of an anti-CD45 antibody capable of inhibiting CD45 phosphatase activity.

Other objectives and advantages of the invention may be apparent to those skilled in the art from a review of the following detailed description, including any drawings, as well as the approved claims.

### BRIEF DESCRIPTION OF THE DRAWINGS

FIGS. 1A-1C demonstrate that CD45 negatively regulates cytokine-induced activation of mast cells and JAKs. FIG. 1A: increased proliferation of cd45^{-/-} BMMCs. cd45^{+/+} and cd45^{-/-} BMMCs were incubated with increasing doses of IL-3 and proliferation was assessed by [³H] thymidine incorporation. Asterisks, P<0.01 between cd45^{+/+} and cd45^{-/-} BMMCs; FIG. 1B: enhanced JAK2 phosphorylation. BMMCs were stimulated with IL-3 (30 ng/ml). JAK2 was immunoprecipitated; this was followed by immunoblotting with anti-phosphotyrosine and anti-JAK2. ERK activation was assayed with an anti-phospho-ERK antibody; FIG. 1C: increased JAK2 kinase activity in cd45^{-/-} BMMCs stimulated with IL-3 (30 ng/ml). JAK2 activity was assessed by kinase assay in vitro. Values (s.e.m.) are expressed as fold increase over control (wild type, no IL3).

FIGS. 2A-2C show that CD45 deficiency leads to increased tyrosine phosphorylation of STATs. FIG. 2A: increased STAT phosphorylation in the absence of CD45. cd45^{+/+} and cd45^{-/-} BMMCs were treated with IL-3 (30 ng/ml) and phosphorylation was determined with phosphospecific antibodies against STAT3 (Tyr 705) (P-Y705-STAT3), STAT3 (Ser 727) (P-S727-STAT3) or STAT5 (Tyr 694) (P-Y694-STAT5); FIG. 2B: increased DNA-binding activity of STAT3 in cd45^{-/-} BMMCs treated with 30 ng/ml IL-3. Nuclear extracts and a radiolabeled STAT3-specific probe were used in EMSA. In the last lane, an excess of the unlabelled DNA probe was added (competition +). The lower panel shows mean STAT3 binding (s.e.m.) from triplicate experiments. FIG. 2C: enhanced induction of cyclin D1 mRNA in the absence of CD45. Induction of cyclin D1 was analyzed by northern blot at the indicated time points after stimulation with IL-3. 28 S rRNA is shown as control. FIGS. 3A-3F demonstrate that CD45 dephosphorylates JAKs in vitro. FIG. 3A: recombinant CD45 dephosphorylates Lyn and JAK2, but not STAT3, in vitro. FIG. 3B: vanadate inhibits rCD45-mediated JAK2 dephosphorylation. Phosphorylation was examined with anti-phosphoryrosine, anti-phospho-Tyr 705 STAT3 (P-Y705-STAT3), and anti-phospho-Tyr 1007/Tyr 1008 JAK2 (P-Y1007/1008-JAK2). Assays were performed with 10 U (for JAK2) or 90 U (for STAT3) of rCD45. FIG. 3C: Decreased kinase activity of JAK2 dephosphorylated by rCD45 (10 U). Asterisks, P<0.001, kinase activity between non-treated and treated with CD45. FIG. 3D: CD45 dephosphorylates JAK1 and Tyk2. FIG. 3E, CD45 binds to JAK2. JAK2 was incubated with GST alone, GST-CD45 (cytoplasmic portion of a PTPase-dead mutant), and GST-D2 (non-catalytic D2 domain of CD45). Proteins bound to JAK2-beads were detected by anti-GST. FIG. 3F: heterotopic expression of CD45 suppresses tyrosine phosphorylation of JAK1. COS cells were transfected with wild-type CD45 (CD45), phosphatase-inactive CD45 (CD45-CS), or mock-transfected controls and stimulated at 48 h with IFN-“TYPE=PICT;ALT=alpha” (500 U/ml). Phospho Tyr 1022/ Tyr 1023 levels of JAK1, total JAK1 and expression levels of transfected CD45 are shown.

FIGS. 4A-C demonstrate that JAKs are hyperphosphorylated in CD45-deficient B cells, thymocytes and Jurkat T cells. FIG. 1A: parental wild-type Jurkat cells (WT), cd45-deficient Jurkat cells (CD45-AS), CD45-AS cells re-expressing wild-type CD45 (CD45-REC) and CD45-AS cells re-expressing PTPase-dead CD45 (CD45-C828S) were stimulated with IFN-″TYPE=PICT;ALT=alpha″ (100 U/ml). FIG. 4B: freshly isolated thyrnocytes from cd45^{+/+} and cd45^{-/-} mice were stimulated with IFN-“TYPE=PICT;ALT=alpha” (20,000 U ml⁻¹). FIG. 4C: freshly isolated splenic B cells from cd45^{+/+} and cd45^{-/-} mice were stimulated with recombinant IL-4 (100 ng/ml) for 10 min. Tyrosine phosphorylation of JAK1 and JAK3 was monitored as described in Example 1.

FIGS. 5A-5D demonstrate that enhanced erythroid colony formation and antiviral activity in the absence of CD45. FIG. 5A: in vitro EPO-dependent erythroid colony-forming ability of cd45 (white columns) and cd45^{-/-} (black columns) bone-marrow progenitors. Mean numbers of erythroid colonies (s.e.m.) were scored at day 7. Asterisks, P<0.01 between cd45 and cd45^{-/-} colonies. FIG. 5B: parental Jurkat cells (green), cd45-deficient Jurkat cells (black), CD45-AS cells re-expressing wild-type CD45 (blue) and CD45-AS cells reconstituted with PTPase-dead CD45 (red) were left untreated (None) or treated with IFN-“TYPE=PICT;ALT=alpha” before the addition of CVB3. Asterisks, P<0.01, wild-type or CD45-REC compared with CD45-AS or CD45-C828S cells. FIG. 5C: survival of CVB3-infected cd45^{-/-} (filled circles) and cd45 (open circles) littermate mice. CVB3-infected cd45 mice showed signs of severe systemic illness, whereas cd45^{-/-} mice showed no signs of disease and had a 100% survival rate even at later time points (>60 days after infection). FIG. 5D: histopathology of hearts of CVB3-infected cd45 mice and cd45^{-/-} littermates (day 35 after initial infection). Masson staining. Original magnification “TYPE=PICT;ALT=times”200.

### DETAILED DESCRIPTION OF THE INVENTION

Proliferation and differentiation of cells is regulated, to a great extent, by the extracellular molecules, antigens and cytokines. These external stimuli (ligands) generally generate cellular response by binding to receptor proteins found in the cell's plasma membrane. Typically, the binding of a ligand to its respective receptor stimulates either protein kinases or protein phosphatases that, through a cascade of biochemical reactions, effect phosphorylation of serine, threonine, or tyrosine residues on specific transcription factors. While kinases operate by adding a phosphate group to certain amino acid residues, phosphatases operate by removing a phosphate group. Importantly, depending on the particular protein substrate, phosphorylation or dephosphorylation can function to either activate or deactivate the protein. This intracellular signaling by external stimuli results ultimately in gene transcription and is generally referred to as signal transduction.

The transmembrane protein tyrosine phosphatase (PTPase) CD45 is a critical regulator of antigen receptor signaling in T and B cells. The primary molecular targets for CD45 are the cytosolic Src family kinases. CD45 functions by removing the phosphate from a phosphotyrosine in the carboxy terminus of Src family kinases, in turn, activating these kinases. CD45 may also down-regulate the kinase activity of Src family members in thymocytes and during integrin-mediated adhesion in macrophages. Thus, CD45 can function as a positive, as well as a negative regulator, of Src family members and Src-mediated cellular responses.

These Src kinases are common components of signaling pathways associated with regulating cell division and differentiation. CD45 is, however, highly expressed in all hematopoietic lineages and at all stages of development. Thus CD45 could regulate other cell types and possibly act on additional substrates. Indeed a novel and unexpected function of CD45 has been identified. Specifically, CD45 has been shown to be a JAK phosphatase and negatively regulated cytokine receptor signaling [see below and Irie-Sasaki et al., Nature 409:349-354, 2001 and Penninger et al., Nature Immunol. 2(5):389-396, 2001].

Cytokines are small proteins made by cells that affect the behavior of other cells by binding to specific cytokine receptors. Many, if not most, cytokines important for immune responses employ the JAK-STAT signaling pathway. As opposed to antigen induced signal transduction, cytokines signal lymphocytes by binding to cytokine receptors and triggering Janus kinases (JAKs) to phosphorlyate and activate signal transducers and activators of transcription (STAT) proteins. Upon binding of a ligand, receptor associated JAKs transactivate each other by phosphorylating tyrosine residues. By binding to the receptor-JAK complex, STATs are then themselves phosphorylated by JAK. Phosphorylation of STATs, via different combinations of JAK and STAT proteins, ultimately leads to translocation of STAT hetero and/or homodimers to the nucleus. In the nucleus, these proteins function as transcription factors by inducing transcription of various genes important for cell growth and differentiation.

While activation of STAT proteins involved in immune response is an important function attributed to JAK kinases, evidence indicates that JAK kinase function may integrate components of various diverse signaling cascades. For instance, it has been shown that JAK kinase function is required for optimal activation, of the Src kinase cascade and the Ras-MAP kinase signaling pathway, along with STAT signaling following the interaction of cytokine or interferon receptors with their respective ligands. Consequently, defects in JAK kinase activity may lead to defects in numerous signaling events, therefore disrupting normal cellular responses. Indeed, defects in the JAK-STAT pathway have already been identified in a number of human diseases, most notably, leukemias, lymphomas, and inherited immunodeficiency syndromes. For this reason, the JAK-STAT signaling pathway has been studied (see Examples set forth below) extensively a potential target for pharmaceutical intervention.

While the transmembrane PTPase CD45 has been characterized as an antigen receptor that activates the Src family of kinases via dephosphorylation, new and unexpected evidence indicates that CD45 also functions as a hematopoietic JAK phosphatase that negatively regulates cytokine receptor signaling (see data present in Examples). Ire-Sasaki et al. [Nature 409:439-354, 2001] showed (see Examples et forth below) that targeted disruption of the CD45 gene leads to enhanced cytokine and interferon receptor-mediated activation of JAKs and STAT proteins. In vitro, CD45 directly dephosphorylates and binds to JAKs. Functionally, CD45 negatively regulates interleukin-3-mediated cellular proliferation, erythropoietin-dependent hematopoisis, and antiviral responses in vitro and in vivo. Consequently, CD45 may regulate the response of cells to developmental signals and also manage functions such as the growth of red and white blood cells or the regulation of viral infections.

Loss of CD45 expression has been reported in greater than ten percent of human patients with acute lymphoblastic leukemia [Ratei et al., Ann. Hematol. 77:107-114, 1998]. CD45 expression is also shown to be lost in patients with Hodgkin's lymphoma and multiple myelomas and, in the case of a child with B lymphoma, a genetic mutation in CD45 was implicated [Kung et al., Nature Med. 6:343-345, 2000; Ozdemirli et al., Cancer 77:79-88, 1996; Ishikawa et al. Lymphoma 39:51-55, 200]. Evidence that CD45 may function as a tumor suppressor in certain experimental systems has also been shown [Baker et al., EMBO J. 19:4644-4654, 2000]. Furthermore, over expression of CD45 in growth factor dependent cell lines often causes growth arrest and/or cell death. All these phenomena, as well as tumorigenesis may be explained by the activity of CD45 as a negative regulator of cytokine receptor signaling. Further characterization of CD45 is needed to understand the temporal and geographic sequences of events that result in antigen and cytokine receptor-mediated responses. Accordingly, future research in this area is likely to focus on how the cellular signals involved in the pathogenesis of various diseases can be modulated. Finally, these studies hold much commercial value given CD45's function and opportunity for manipulation by designing drugs that turn antigen and cytokine receptor signaling on or off in cancer and autoimmunity.

As set forth in the examples below, CD45 was shown in vivo and in vitro to function as a hematopoietic JAK phosphastase that negatively regulates cytokine receptor-mediated signaling. Experiments designed to identify whether CD45 could dephosphorylate tyrosine-phosphorylated proteins implicated in the development and activation of hematopoietic cells, showed that CD45 directly dephosphorylated and inactivated JAK family members, JAK1, JAK2, and Tyk2, in vitro. Although all sites of JAK dephosphorylation by CD45 have not yet been identified, CD45 is shown by this disclosure to dephosphorylate critical and conserved tyrosine residues in the activation loop of JAK1, JAK2, and Tyk2, which results in decreased JAK activity. The second “pseudo-PTPase” domain of CD45 (D2-domain) physically associates with the JAKs, probably through their second noncatalytic kinase domain. The activation of other cytokine receptor-triggered signaling pathways including extracellular signed-regulated kinases (ERKs) and protein kinase B (PKB)/Akt, was normal in the absence of CD45, implying that CD45 specifically inhibits JAKs and JAK-regulated signaling. Consequently, CD45 may further control JAK-regulated STAT phosphorylation, DNA-binding activity, and gene transactivation.

Through autophosphorylation or transphosphorylation, JAKs associated with the cytoplasmic tails of cytokine receptors are activated upon binding of cytokines to their receptors (cite 33 of man2). JAKS and STATs, can also, however, be hyperphosphorylated in Src-transformed fibroblasts and expression of STATs might be important for transformation of myeloid cells and fibroblasts (Cite 34,35 man2). Because Src family kinases can be activated in response to growth factor receptors, they could possibly contribute to the observed hyperphosphorylation of JAKs and STATs in CD45^{-/-} cells. However, as described in the examples below, Src-family kinases were not activated in CD45^{+/+} or CD45^{-/-} mast cell lines in response to IL-3 nor did inhibition using the PP2 inhibitor of Src family kinases change the increased JAK phosphorylation and JAK activities in CD45^{-/-} cells. While it cannot be excluded that subtle changes in Src kinase activities could contribute to hyperactivation of JAKs and STATs in CD45^{-/-} cells, the results indicate that CD45 can directly deactivate JAK-STAT signaling pathways independent of CD45-regulated Src family kinases.

In view of the foregoing background and discussion, anti-CD45 antibodies capable of activating CD45 phosphatase activity would consequently inhibit JAKs, which in turn would be useful to check unwanted proliferation of hematopoietic cell lineages (all hematopoeitic cells; erythrocyte and platelet progenitors have CD45 and JAKs, thus via CD45 one could positively or negatively control for example platelets for bleeding, erythrocyte regeneration in anemia, white blood cell generation following chemotherapy, T and B cells for proliferation in autoimmune disease, leukemia cell proliferation, proliferation and development of macrophages and neutrophil cells in inflammation/autoimmunity/arthritis/diabetes or mast cells in allergy) or counterbalance cytokine function in many pathophysiological processes such as inflammatory diseases (for example, but not limited to allergic reactions, arthritis, allergy, diabetes, MS, lupus, thyreoditis, myocarditis and artheriosclerosis, chronic and acute bacterial and viral infections such as HIV/Hepatitis).

Conversely, anti-CD45 antibodies capable of inhibiting CD45 phosphatase activity would consequently not inhibit JAKs but activate JAKs (and in some instances cause hyperactivation of JAKs), which would be useful as an alternative way to positively control certain cytokine and growth factor receptors (for example, but not limited to erythropoietin-receptor, IL3-Receptor, c-kit receptor, IL4 receptor, IL2 receptor, IL7 receptor, Interferon alpha-receptor, Interferon beta-receptor, Interferon gamma receptor, and all other receptors that are expressed on hematopoeitic cells that use the JAK-STAT signaling pathway). Moreover, the CD45 homolog LAR (see Examples below) would be capable of acting on the JAK-STAT pathway in non-hematopoeitic cells to help control (for example, but not limited to) diabetes (insulin receptor), breast cancer growth (prolactin-receptor), and obesity (OB-receptor).

In general, the present invention would be useful to (for example, but not limited to) block transplant rejection, block allergic reactions, block acute and chronic inflammatory diseases, enhance regeneration of all hematopoietic cells, and change osteoclast functions in local or systemic bone loss (osteoporosis).

Further aspects of the invention and embodiments will be apparent to those skilled in the art. In order that the present invention be fully understood, the following examples are provided by way of exemplification only and not by way of limitation.

Example 1 provides methods and materials for the subsequent Examples.

Example 2 describes results showing CD45's involvement in cytokine receptor signaling.

Example 3 describes experiments to assess the importance of CD45 on the regulation extracellular signed-regulated kinases.

Example 4 describes the interaction of Src kinases and the hyperactivation of the JAK-STAT pathway.

Example 5 details the direct dephosphorylation of JAKS and/or STATS by CD45.

Example 6 describes results of experiments designed to evaluate whether INF-α induced activation of JAK1 and Tyk2 in increased in CD45-deficient cells.

Example 7 describes the effects of a CD45 deficiency on Cytokine-dependent erythropoiesis and myelopoiesis.

Example 8 described the effects of a JAK-STAT mediated response to viral infections.

Example 9 describes methods for the production of antibodies that may either activate or inhibit the action of CD45.

### EXAMPLE 1

Methods and Materials

Methods and materials used or referred to in subsequent examples are set forth directly below.

Mice, Cells and Reagents

CD45-exon6 [Tonks et al., Cell 87:365-368, 1996] and CD45-exon9 [Byth et al., J. Exp. Med. 183:1707-1718, 1996] mice have been previously generated and were maintained in accordance with institutional guidelines. BMMCs were cultured as described [Lui et al., Genes Dev. 13:786-791, 1999]. Macrophages, thymocytes and splenic B cells were freshly isolated from cd45 wild-type and cd45-mutant littermate mice [Lui et al., Genes Dev. 13:786-791, 1999]. The CD45-negative human Jurkat cell lines CD45-AS (J-AS-1, McKenney et al., J Biol. Chem. 270:24949-24954, 1995] and J45.01 were cultured in RPMI medium plus 5% FCS (fetal calf serum). Phycoerythrin-, fluorescein isothiocyanate- or biotin-conjugated antibodies against pan-CD45, CD19, c-Kit (CD119), Gr1 and Mac1 (CD11b) (PharMingen) were used for flow cytometry. Antibodies against STAT3, STAT1, STAT5, or PKB/Akt and STAT3 (Tyr 705), STAT3 (Ser 727), STAT5 (Tyr 694), STAT1 (Tyr 701), PKB/Akt (Ser 473 or Thr 308), ERK1 ERK2 (Thr 202 and Tyr 204), JAK1 (Tyr 1022 and Tyr 1023), Tyk2 (Tyr 1054 and Tyr 1055) and JAK2 (Tyr1007 and Tyr 1008) were from QCB. The anti-phosphotyrosine (PY99) antibody and antisera against Lyn, JAK1, JAK2, Tyk2, JAK3 and c-Kit were from Santa Cruz Biotechnology. PP2, recombinant CD45, LAR, TC-PTP and lambda phosphatase were from Calbiochem. Antibodies against JAK3 and IL-3 receptor β-chain and purified JAK2 and Lyn were from Upstate Biotech. One unit of phosphatase activity was defined as the amount of enzyme that hydrolysed 1.0 nmol of p-nitrophenyl phosphate per minute at 30° C. and pH 7.0.

Immunoblotting, Immunoprecipitation and Transient Expression of CD45

Murine BMMCs, splenic B cells, thymocytes, peritoneal macrophages and Jurkat T-cell lines were stimulated with various cytokines. For immunoprecipitations, lysates were incubated with antibodies conjugated with agarose or Sepharose beads for 2 h at 4° C. Total cell lysates or immunoprecipitates were separated using SDS-polyacrylamide gel electrophoresis, transferred onto membranes and immunoblotted. COS cells were transfected using diethylaminoethyl-dextron transfection technique with wild-type or PTPase-dead CD45 (D1:C828s) as described [McKenney et al., J. Biol. Chem. 270:24949-24954, 1995].

Assays

For tyrosine phosphatase assays, immunoprecipitated Lyn, JAKs or STATs from lysates of 10⁷ BMMCs (for Lyn, JAK1, JAK2, STAT3 and STAT5) or macrophages (for Tyk2) were washed in lysis buffer and phosphatase reaction buffer [Felberg et al., J. Biol. Chem. 273:17839-17845, 1998]. The amounts of Lyn and JAK2 in the immunoprecipitates used in FIG. 3A were 72 ng and 48 ng, respectively, as determined by silver staining. Recombinant CD45 was added to substrates in the presence or absence of 0.1 mM Na₃ VO₄ followed by incubation at 30° C. for 20 min. Tyrosine phosphorylation was monitored by immunoblotting with phosphotyrosine-specific antibodies. The extent of tyrosine phosphorylation and amount of substrates were determined by quantifying the intensities of the immunoblots with NIH Image 1.61 software. Kinase activity in anti-Lyn or anti-JAK immunoprecipitates was assessed by an in vitro kinase assay with Raytide (Oncogen Science) as a substrate or using authophosphorylation [Lui et al., Genes Dev. 13:786-791, 1999]. In vitro binding assays were performed as described [Felberg et al., J. Biol. Chem. 273:17839-17845, 1998], using 1 μg of soluble GST, GST-CD45-D2, or GST-PTPase-dead CD45 (D1:C828S). EMSAs were as described [Charurvedi et al., Mol. Cell. Biol. 17:3295-3304, 1997]. The sequences of the EMSA probes were: STAT3, 5′-GATCCTTCTGGGAATTCCTAGATC-3′ (SEQ ID NO: 1); STAT3 mutant, 5′-GATCCTTCTGGGCCGTCCTAGATC-3′ (SEQ ID NO: 2).

Proliferation, Cell Death, Colony Formation and CVB3 Infections

For proliferation of BMMCs, cells were deprived of IL-3 for 12 h and then stimulated with IL-3 for 24 h and pulse-labeled with [³H]thymidine for 12 h. The percentage of cell death was determined by 7-amino-actinomycin D (7AAD) and staining with Annexin V. Hematopoietic precursors were isolated from the bone marrow of 6-week-old cd45^{Γ/Γ} and cd45^{+/+} mice and the formation of EPO-dependent erythroid colonies (BFU-E) and IL-3-dependent mixed neutrophil/macrophage myeloid colonies was determined. Total bone-marrow cells were plated in methylcellulose containing 5% fetal bovine serum. EPO (0.1, 0.3 and 1 unit ml^{Γ1}) or IL-3 (0.1 ng ml^{Γ1}) was added to the culture to induce erythroid or myeloid colony formation, respectively. The sizes and numbers of colonies were analyzed 7 and 12 days after the start of culture.

Virus Infections

For Coxsackievirus infections in vitro, Jurkat cells were pretreated with IFN-α (1,000 units ml−¹) for 4 h and inoculated in triplicate with 3×10⁵ CVB3 for 1 h at 37° C. Cells were then washed to remove excess IFN-α and excess CBV3 virus, and resuspended in RPMI medium containing 10% FBS. After 24 h, culture supernatants were analyzed for the presence of infectious CVB3 by using plaque-forming assays in HeLa cell monolayers. For CVB3 infections in vivo , the cd45mutation was backcrossed onto an A/J (H2^{k/k} ) background for five generations. A/J (H2^{k/k}) mice are highly susceptible to acute and chronic CVB3 infections. Four-week-old mice were inoculated intraperitoneally with 10⁵ plaque-forming units of CVB3. For lethality scores, cd45−^{/-} (n=25) and cd45^{±} (n=22) littermate mice were monitored daily after infection. CVB3-infected cd45^{±} mice showed signs of severe systemic illness, including lethargy, ruffled coats and anorexia, before death, but cd45−^{/-} mice failed to show these signs. At defined time points, mice were sacrificed and organs were processed for histology. Sections from eight mice per group were stained with haematoxylin/eosin and Masson blue. In all experiments, the CVB3 strain Gauntt-Chow was used.

### EXAMPLE 2

CD45 Involvement in Cytokine Receptor Signaling

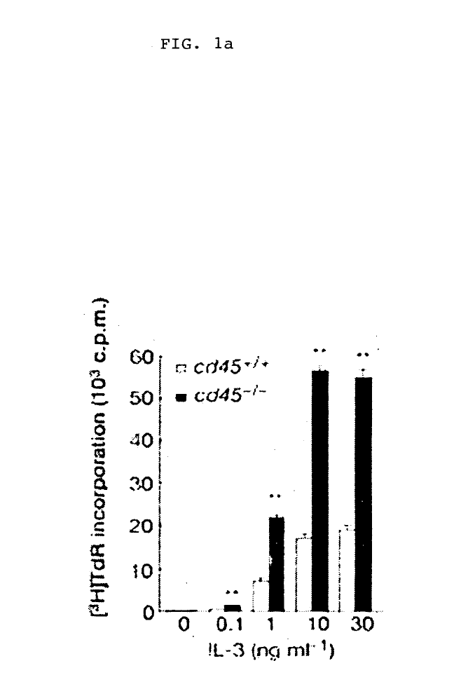

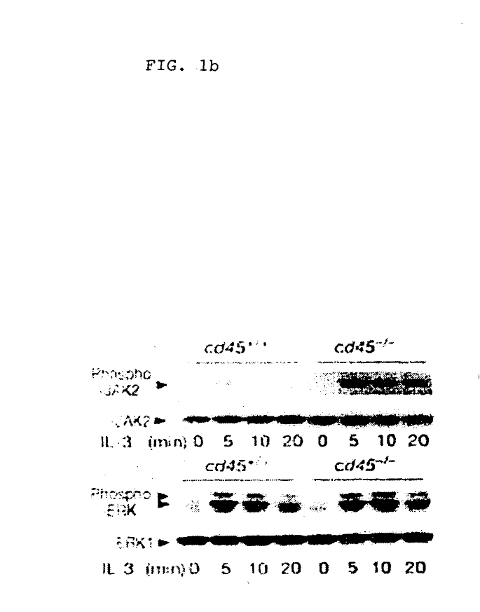

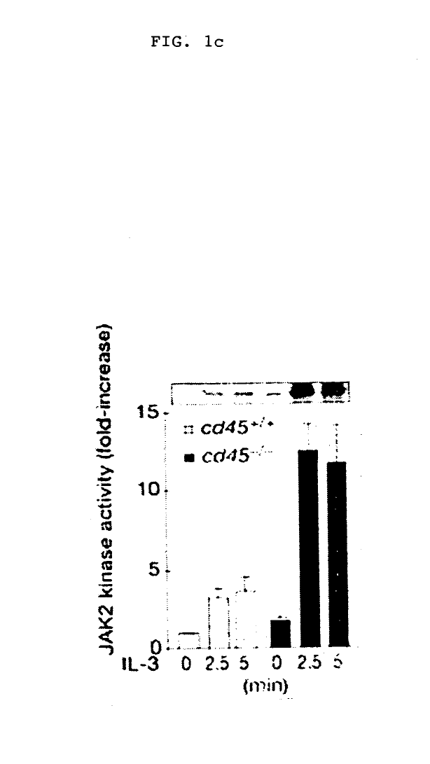

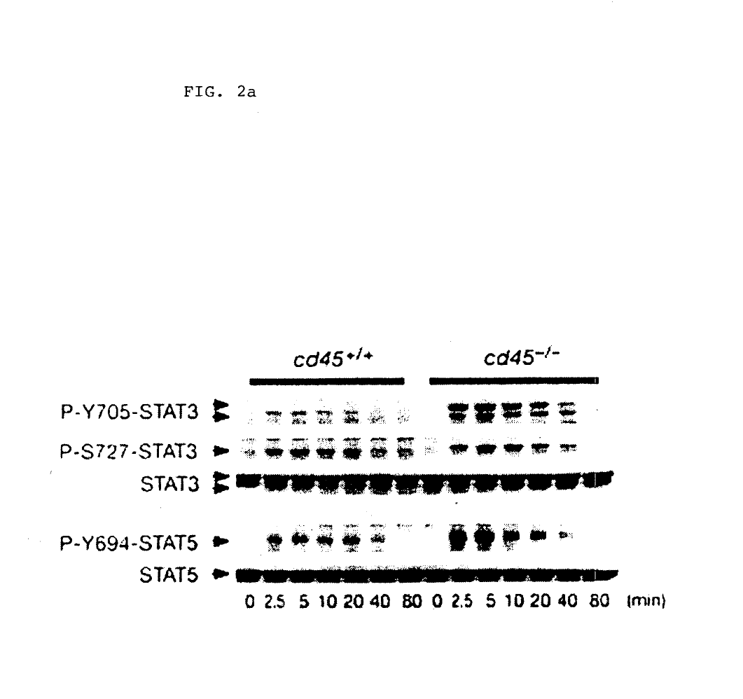

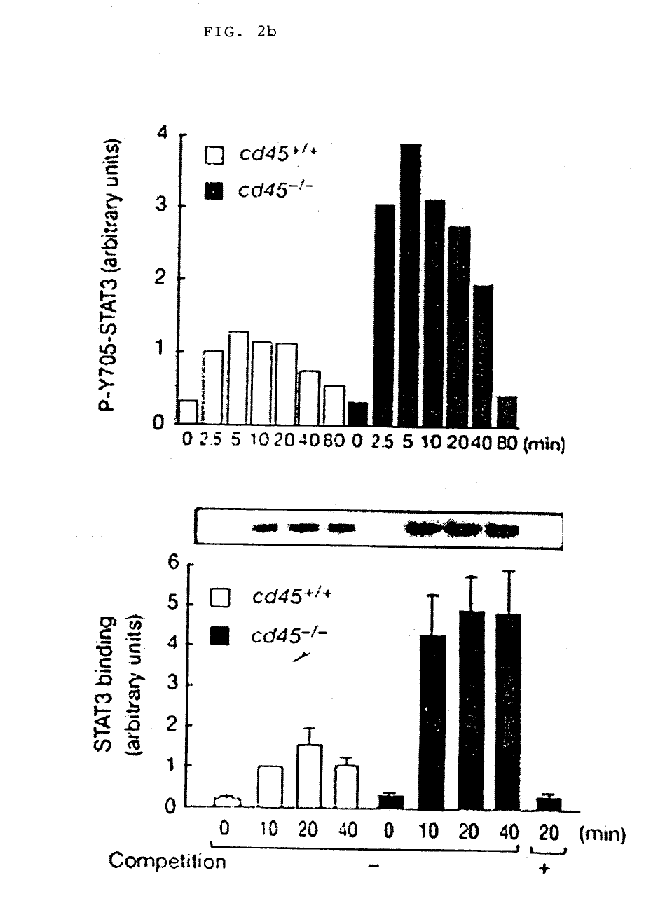

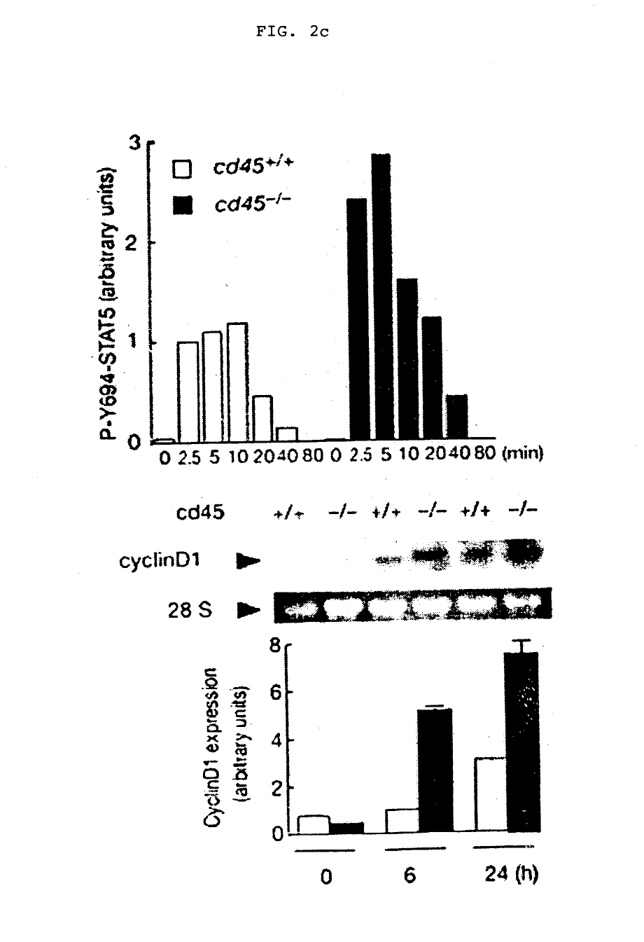

Experiments were conducted to assess the involvement of CD45 in cytokine receptor signaling. Specifically, interleukin-3 (IL-3)-dependent bone-marrow-derived mast cell (BMMC) lines were derived from cd45^{+/+} mice and mice that carry a mutation in exon 6 of the cd45 gene (cd45^{-/-} [Kishihara et al., Cell, 74:143-156, 1993]. BMMCs from both genotypes showed similar expression levels of FcεR1, c-Kit, and IL-3 receptor β-Chain, indicating that a lack of CD45 does not prevent the emergence and differentiation of BMMCs. Results of generating the mast cells with IL-3 indicated that the expansion rate of cd45^{-/-} BMMCs was greater than that of cd45^{+/+} BMMCs. Proliferation of cd45^{-/-} BMMCs was markedly increased in response to stimulation with IL-3 over that of wild-type cells (FIG. 1A). Thus, in contrast to antigen receptor stimulation, in which a loss of CD45 leads to decreased cell growth [Kishihara et al., Cell, 74:143-156, 1993; Byth et al., J. Exp. Med. 183:1707-1718, 1996], our data show that a loss of CD45 expression leads to an increased proliferation of BMMCs in response to the cytokine IL-3.

### EXAMPLE 3

CD45 Regulation of Additional IL-3 Triggered Signaling Pathways

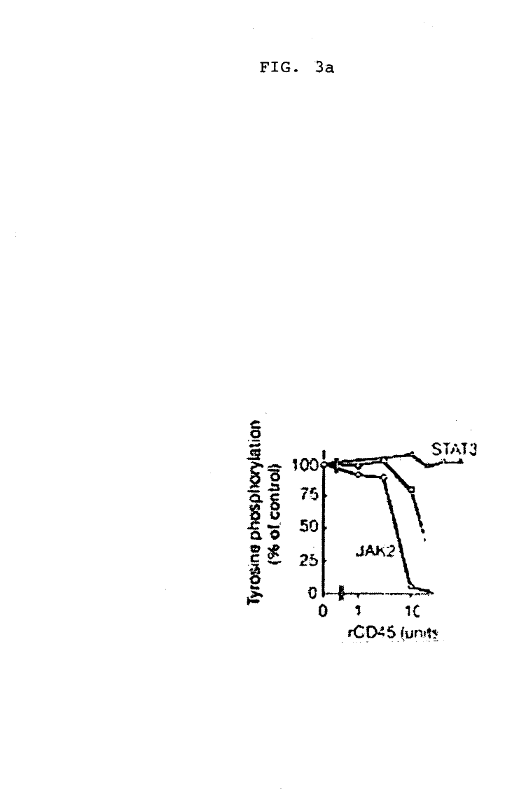

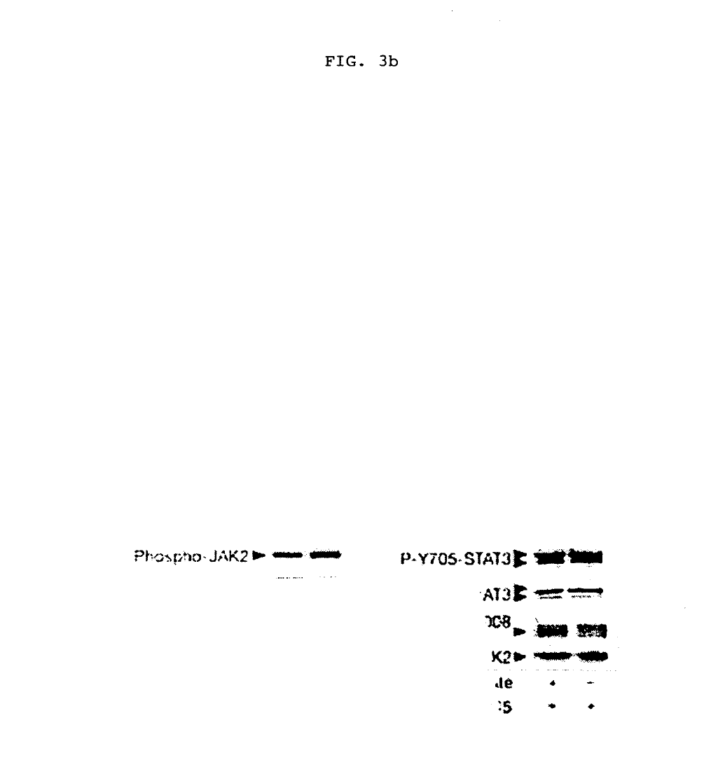

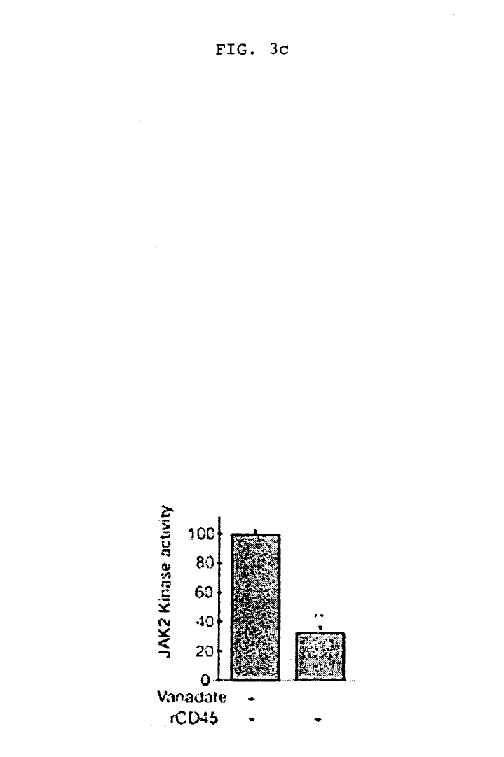

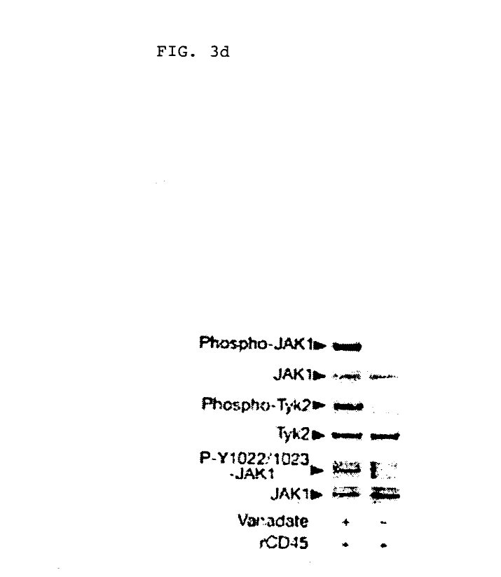

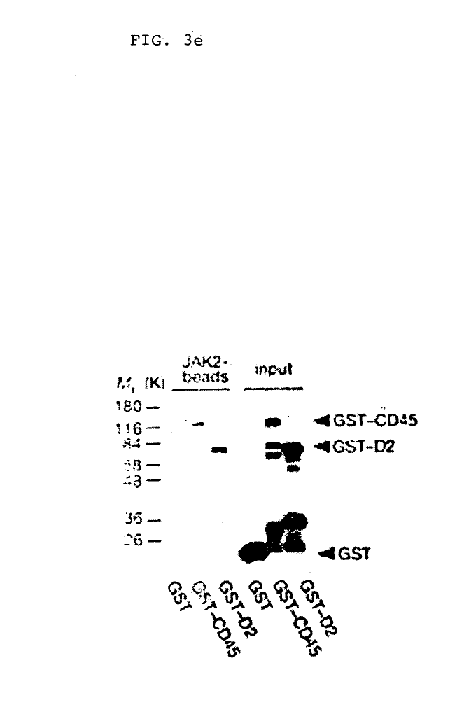

Because stimulation with IL-3 triggers multiple signaling pathways including the activation of extracellular signed-regulated kinases (ERKs), protein kinase B (PKB)/Akt and JAKs [de Groot et al., Cell Signal. 10:619-628, 1998], experiments were designed to assess the importance of CD45 on regulation of these signaling molecules. Specifically, phosphorylation of these molecules in response to IL-3 was measured. BMMCs were stimulated with IL-3 (30 ng ml−1). Following immunoprecipitation of JAK2, immunoblotting with anti-phosphotyrosine and anti-JAK2 was performed. ERK activation was assayed with an anti-phospho-ERK antibody. The results obtained indicated that phosphorylation of ERK1/ERK2 (FIG. 1B) and phosphatidylinositol-3-OH kinase (P1(3)K)-dependent activation of PKB/Akt were comparable between wild-type and cd45-deficient BMMCs. In cd45^{+/+} BMMCs, Jak2 tyrosine phosphorylation in response to IL-3 was readily detected. Surprisingly, IL-3-induced JAK2 tyrosine phosphorylation was significantly enhanced in cd45^{-/-} BMMCs (FIG. 1B). Tyrosine phosphorylation of JAK2 was also enhanced in BMMCs derived from a line of independently derived CD45-null mice [Byth et al., J. Exp. Med. 183:1707-1718, 1996]. Consistent with JAK2 hyperphosphorylation was the observation that JAK2 kinase activity was increased in the absence of CD45 after stimulation with IL-3 [FIG. 1C]. JAK 2 activity was assessed by kinase assay in vitro. Values are expressed as fold increase over control (wild-type, no IL-3). In addition, tyrosine phosphorylation of the JAK substrates STAT3 (Tyr 705) and STAT5 (Tyr 694) in response to IL-3 was significantly increased in cd45^{-/-} BMMCs in comparison with wild-type BMMCs [FIG. 2A]. After treating cd45^{+/+} and cd45^{-/-} BMMCs with IL-3 (30 ng ml−1), phosphorylation was determined with phosphospecific antibodies against STAT3 (Tyr 705) (P-Y705-STAT3), STAT3 (Ser 727) (P-S727-STAT3) or STAT5 (Tyr 694) (P-Y694-STAT5). Phosphorylation of STAT3 on Ser 727 in response to IL-3 was comparable in cd45^{+/+} and cd45^{-/-} BMMCs [FIG. 2B ], indicating that the PTPase CD45 does not regulate the serine phosphorylation of STAT3. Phosphorylation of Tyr 705 in STAT3 and Tyr 694 in STAT5 is required for the dimerization and transcriptional activity of these STATs [Leonard et al., Ann. Rev. Immunol. 16:293-32, 1998 ]. DNA binding of STAT3 was also analyzed. Nuclear extracts and a radiolabeled STAT3-specific probe were used in EMSA. Consistent with the enhanced phosphorylation of STAT3, was the observation that stimulation with IL-3 induced more DNA-binding activity of STAT3 in BMMCs lacking CD45 than in control cd45^{+/+} BMMCs [FIG. 2B]. In addition, cyclin D1 induction was evaluated using northern blot analysis. The induction of cyclin D1, which mediates IL-3-dependent proliferation through STAT5, was enhanced in cd45^{-/-} BMMCs {FIG. 2C]. These results indicate that CD45 negatively regulates the IL-3-triggered JAK-STAT signaling pathway.

### EXAMPLE 4

Src Kinases and the Hyperactivation of the JAK-STAT Signaling Pathway

Experiments were designed in order to assess whether Src kinases, known substrates of CD45, were deregulated during hyperactivation of the JAK-STAT pathway in cd45^{-/-} BMMCs. Using the Src family kinase inhibitor PP2 [Hanke et al., J. Biol. Chem. 271:695-701, 1996], the results showed that JAK2 activation was unaffected by addition of the inhibitor in both the cd45^{+/+} and cd45^{-/-} BMMCs. In addition, in IL-3 stimulated cd45^{+/+} or cd45 ^{-/-} BMMCs, treatment with PP2 had no effect on the increased phosphorylation of the specific residues Tyr 1007 and TYR1008 in JAK2, Tyr 705 in STAT3, and Tyr 1022 and Tyr 1023 in JAK1 (see FIG. 2). These results indicate that Src-family kinases do not account for increased JAK-STAT activation in IL-3 stimulated mast cells.

### EXAMPLE 5

Direct Dephosphorylation of JAKs and/or STATs by CD45

To determine whether CD45 could directly dephosphorylate JAKs and/or STATs, phosphatase assays were used in in vitro studies with recombinant CD45. The rCD45 employed contained the intracellular dual PTPase domains with a relative molecular mas of 97,000 (Mr 97K) (the catalytic D1 and the non-catalytic D2 domains) of CD45 but lacked its extracellular regions. As expected, rCD45 dephosphorylated Lyn in a dose-dependent manner (FIG. 3A). In contrast, tyrosine dephosphorylation of STAT3 (FIGS. 3A and 3B) and STAT5 did not occur after incubation with rCD45, even at high concentrations of rCD45. rCD45 did, however, dephosphorylate JAK2 in vitro (FIG. 3A). In contrast, the human non-receptor tyrosine PTPase TC-PTP and the bacterial lambda PTPase did not dephosphorylate JAK2 in vitro. Phosphorylation was examined with anti-phosphoryrosine, anti-phospho-Tyr 705 STAT3 (P-Y705-STAT3), and anti-phospho-Try 1007/Try 1008 JAK2 (P-Y1007/1008-JAK2). Assays were performed with 10U (for JAK2) or 90U (for STAT3) of rCD45. Incubation of phosphorylated JAK2 with rCD45 in the presence of the PTPase inhibitor mandate blocked JAK2 dephosphorylation [FIG. 3B]. Lastly, PTPase-dead rCD45 (D1:C828S mutant) [Felberg et al., J. Biol. Chem. 273:17839-17845, 1998] did not dephosphorylate JAK2, demonstrating that CD45 PTPase activity is responsible for JAK2 dephosphorylation.

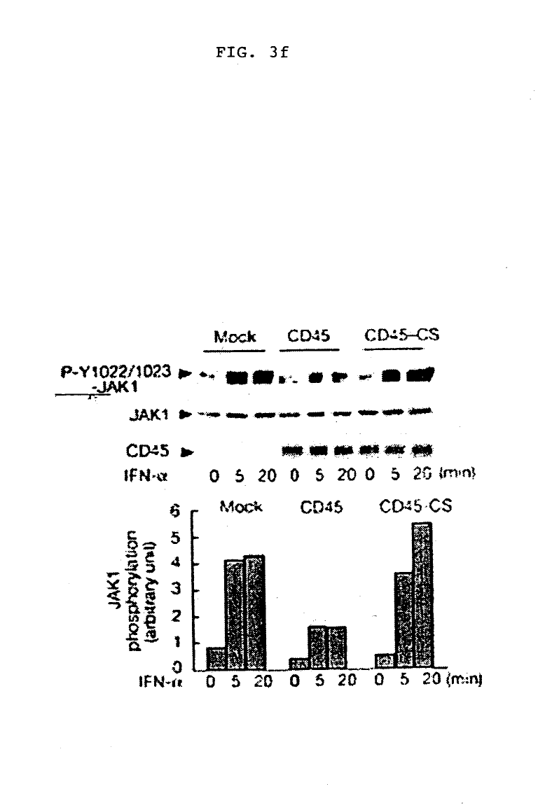

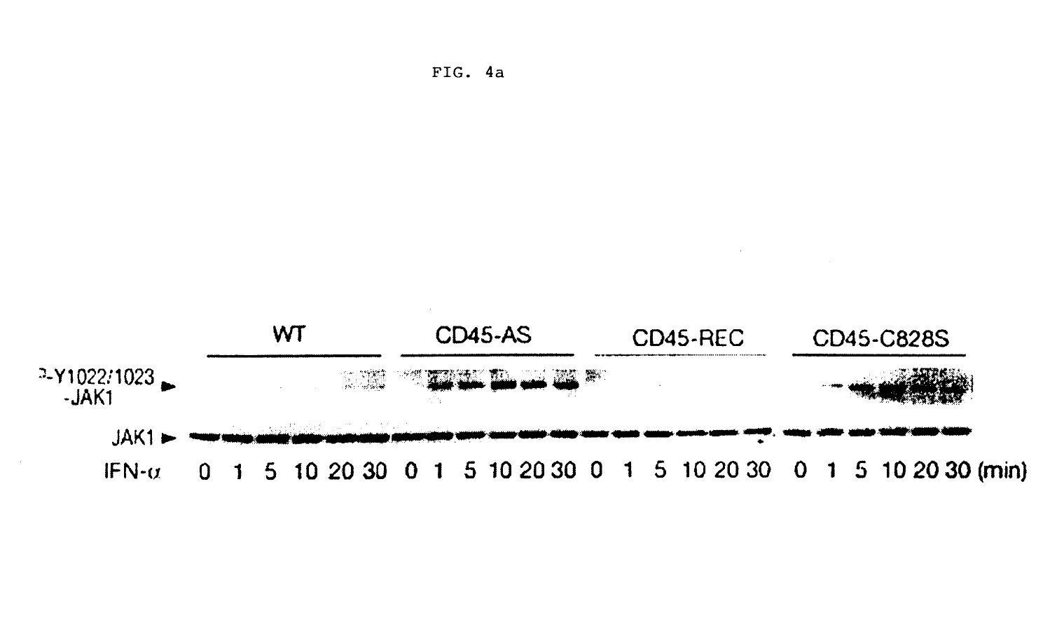

Experiments were also designed using site-specific and phospho-specific antibodies to determine whether the critical tyrosine residues of JAKs are dephosphorylated by rCD45 (FIGS. 3B, 3C, and 3D). The results obtained show that rCD45 does indeed dephosphorylate the critical tyrosine residues of JAK1, JAK2, and Tyk2 in vitro. Notably, as with the phosphatase assays in vitro, the critical tyrosine residues of JAK1, JAK2, and Tyk2 are hyperphosphorylated in cd45-deficient cells (FIG. 4A). In addition, a physical association between JAK2 and the intracellular portion of a PTPase-inactive CD45 trap mutant (glutathione S-transferase (GST)-CD45 D1:C828S). In additional experiments, JAK2 was incubated with GST alone, GST-CD45 (cytoplasmic portion of a PTPase-dead mutant), and GST-D2 (non-catalytic D2 domain of CD45. Proteins bound to JAK2-beads were detected by anti-GST. Notably, the second ‘pseudo-PTPase’ domain of CD45 (GST-D2) could itself bind to JAK2 (FIG. 3E).

To further examine whether the PTPase activity of CD45 is required for the negative regulation of JAKs in vivo, experiments were designed where wild-type and PTPase-dead CD45 (D1:C828S) were expressed in CD45-negative COS cells (FIG. 3F). The results indicated that stimulation of COS cells with interferon-α (IFN-α) triggers the tyrosine phosphorylation of JAK1 at Tyr 1022 and Tyr 1023. Ectopic expression of wild-type CD45 resulted in decreased JAK1 tyrosine phosphorylation (FIG. 3F). Importantly, expression of the PTPase-inactive point mutant of CD45 had no effect on JAK1 tyrosine phosphorylation in response to IFN-α. Therefore, CD45 can associate with JAK2 and directly regulate the tyrosine phosphorylation of JAK-family kinases.

### EXAMPLE 6

### IFN-A Activation of JAK1 AND TYK2 in CD45−/− Jurkat T Cells

A wide variety of cytokines are known to activate JAKs [Leonard et al., Ann. Rev. Immunol. 16:293-322, 1998 and Ihle, J. N., Nature, 377:591-594, 1995]. For this reason, experiments were designed to evaluate whether IFN-α-induced activation of JAK1 (FIG. 4A) and Tyk2 is increased in cd45-deficient (CD45-AS) human Jurkat T cells in comparison with the parental wild-type Jurkat cells (FIG. 4A). Specifically, parental wild-type Jurkat cells (WT), cd45-deficient Jurkat cells (CD45-AS), CD-AS cells re-expressing wild-type CD45 (CD45-REC) and CD45-AS cells re-expressing PTPase dead CD45 (CD45-C828S) were stimulated with IFN-α (100 U ml−1). The results obtained demonstrate that re-expression of wild-type CD45 in the CD45-AS cells decreased the levels of JAK1 phosphorylation to that of the parental Jurkat cells. Importantly, re-expression of PTPase-dead CD45 (D1:C828S) in CD45-AS cells did not alter increased JAK1 phosphorylation (FIG. 4A). Thus, as in the COS cell experiments, CD45-PTPase activity is required for the negative regulation of JAK1 phosphorylation in IFN-α-activated Jurkat cells. In addition, stimulation with IFN-α of freshly isolated thymocytes resulted in JAK1 phosphorylation that was higher in cd45^{-/-} cells than in wild-type thymocytes (FIG. 4B). Furthermore, JAK1 and JAK3 tyrosine phosphorylation was upregulated in primary cd45^{-/-} B cells in response to the B-cell cytokine IL-4 (FIG. 4C). Lastly, in freshly isolated macrophages from cd45^{-/-} mice, Tyk2, STAT1 (Try 701) and STAT3 (Try 705), but not ERK1/2, were hyperphosphorylated in response to IFN-α.

### EXAMPLE 7

Effect of CD45 Deficiency on Cytokine-Dependent Erythropoiesis and Myelopoiesis

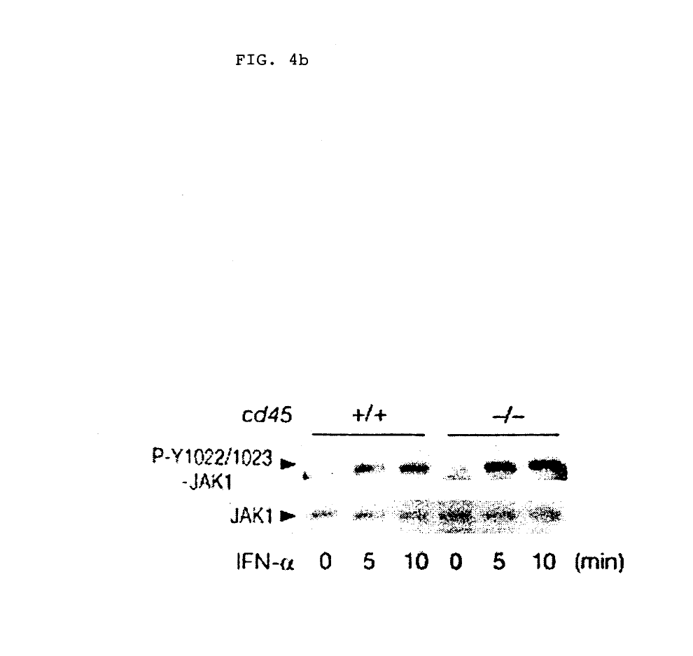

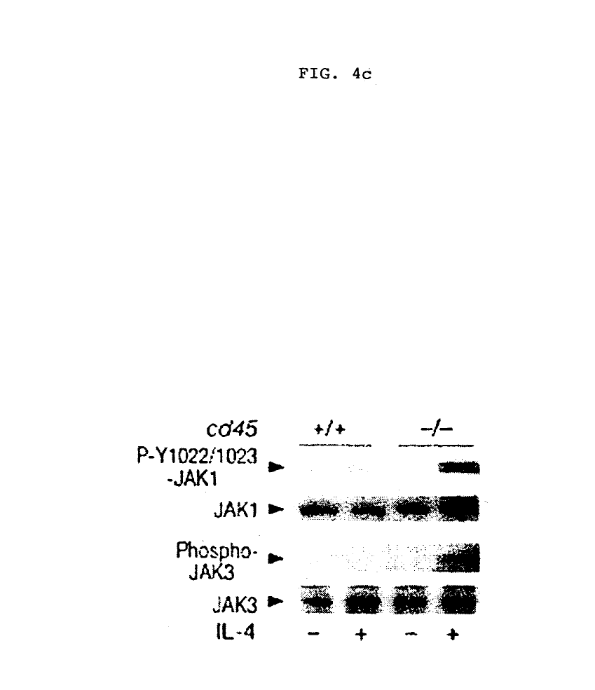

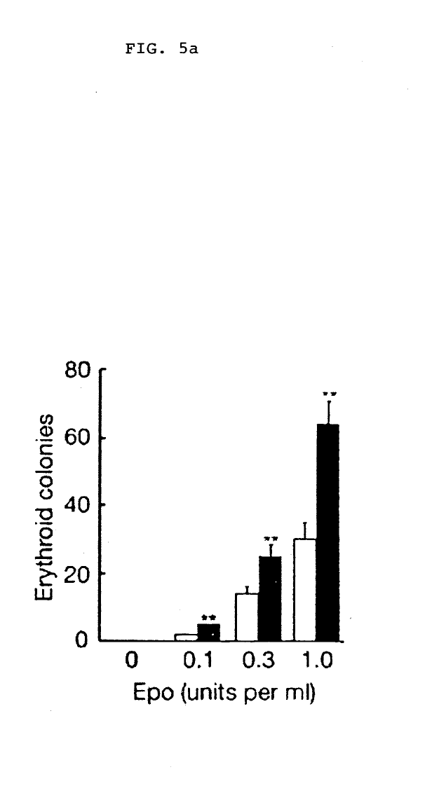

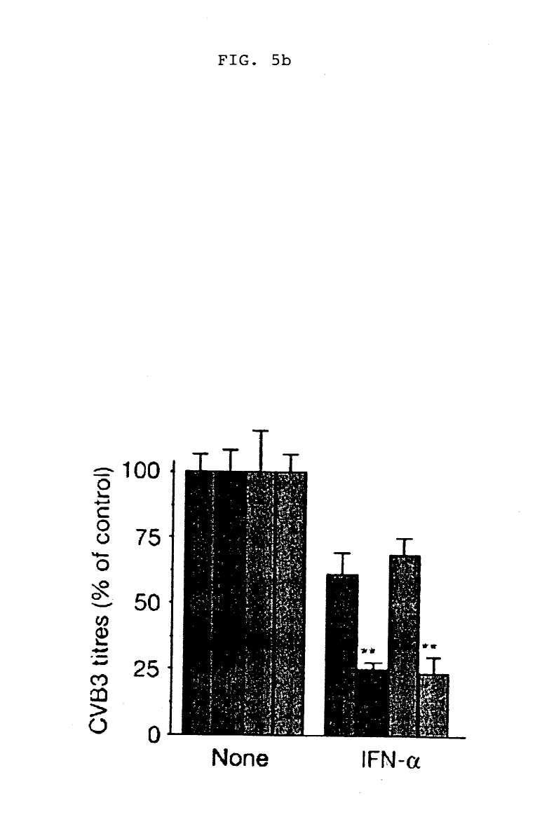

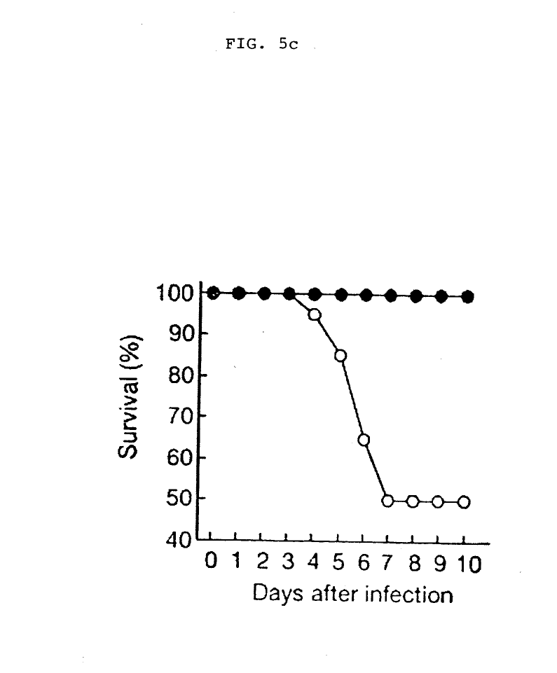

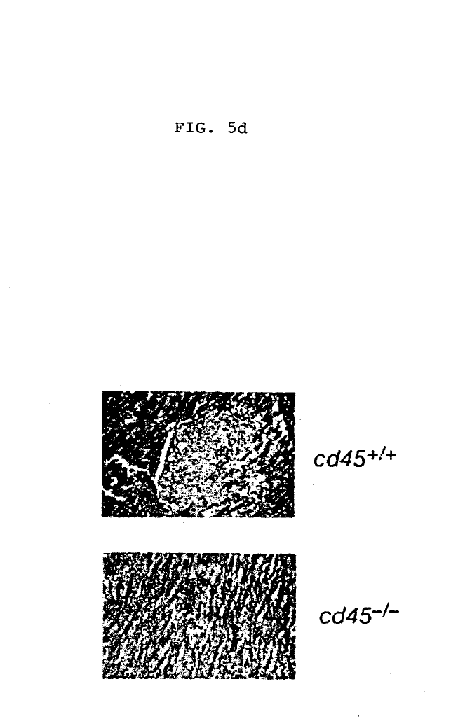

Because cytokine-mediated activation of JAKs is important for a wide array of cellular functions, experiments were designed to investigate the effect of CD45 deficiency on cytokine-dependent erythropoiesis and myelopoiesis. The addition of erythropoietin (EPO) to bone-marrow progenitors induces the growth and differentiation of erythroid colonies (BFU-Es; burst-forming units-erythroid) in a dose-dependent manner [Paraganas et al., Cell, 93:385-395, 1998 and Marine et al., Cell 98:617-627, 1999]. The numbers of EPO-dependent erythroid colonies that differentiated from cd45^{-/-} progenitors were significantly increased in comparison with the numbers derived from cd45^{+/+} progenitor cells (FIG. 5A). Individual cd45^{-/-} erythroid colonies were also substantially greater in size and density than EPO-dependent cd45^{+/+} erythroid colonies. In addition, the formation of IL-3-induced mixed neutrophil/macrophage myeloid colonies was significantly increased when bone-marrow progenitors were isolated from cd45^{-/-} mice as opposed to cd45^{+/+} littermates (cd45^{+/+}, 139±20; cd45^{-/-}, 206±29 (P<00.1; 0.1 ng/ml IL-3, determined on day 7). Thus, a loss of CD45 in hematopoietic progenitor cells leads to increased cytokine-dependent erythropoiesis and myelopoiesis.

### EXAMPLE 8

JAK-STAT Mediated Response to Viral Inffections Through IFN-α and IFN-α Receptor (IFN-αR) Activation

IFN-α and IFN-α R-regulated signaling are critical for replication of the picomavirus Coxsackievirus B3 (CVB3) in vitro and CVB3 infections in vivo [Kandolf et al., J. Mol. Cell. Cardiol. 17:167-181, 1985]. For this reason, experiments were designed to test whether DE45 can regulate DVB3 replication directly in vitro. CVB3 titres were measured after infection of parental or cd45-deficient Jurkat cell lines. IFN-α suppressed viral amplification more efficiently in cd45-deficient cells than in parental Jurkat cells. This suppression was dependent on a functional CD45-PTPase domain (FIG. 5B). To determine whether reduced CVB3 replication in cd45-deficient Jurkat cell lines would translate into altered disease pathogenesis of CVB3 infections in vivo, cd45^{±} and cd45^{-/-} littermate mice were inoculated with CVB3. This treatment was fatal to approximately 50% of the cd45^{±} mice within 7 days of infection. Fatality was due to acute cytopathic effects of the virus leading to encephalitis, pancreatitis, myocarditis, and hepatitis. Alternatively, the cd45^{-/-} littermates were completely protected from the lethal CVB3 infections (FIG. 5C) and unlike the cd45^{±} mice, did not show any histological lesions of acute or chronic inflammation in the heart, pancreas, liver, or brain (FIG. 5D).

### EXAMPLE 9

Production of Antibodies to CD45

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., Immunol Today 4: 72 (1983)) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., Proc Natl Acad Sci USA 80: 2026-2030 (1983)) or by transforming human B-cells with Epstein Barr Virus in vitro (see Cole, et al., 1985 In: MONOCLONAL ANTIBODIES AND CANCER THERAPY, Alan R. Liss, Inc., pp. 77-96). In addition, human antibodies can also be produced using additional techniques, including phage display libraries (Hoogenboom and Winter, J. Mol. Biol.₁₃ 227:381 (1991); Marks et al., J. Mol. Biol. 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368:812-13 (1994)); Fishwild et al, (Nature Biotechnology 14, 845-51 (1996)); Neuberger (Nature Biotechnology 14:826 (1996)); and Lonberg and Huszar (Intern. Rev. Immunol. 13:65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse® as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., Science 246:1275-1281 (1989)) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J. 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh, et al., Methods in Enzymology 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full-length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby, et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lyrnphocytes against human breast tumor targets.

Various techniques for making and isolating bispecific antibody fragments directly from recombinant cell culture have also been described. For example, bispecific antibodies have been produced using leucine zippers. Kostelny, et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber, et al., J. Immunol. 152:5368 (1994).

Antibodies with more than two valencies are contemplated. For example, trispecific antibodies can be prepared. Tutt, et al., J. Immunol. 147:60 (1991). Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (Fc_R), such as Fc_RI (CD64), Fc_RII (CD32) and Fc_RIII (CD16) (what are these symbols supposed to be?) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

It can be desirable to modify the antibody of the invention with respect to effector function, so as to enhance, e.g., the effectiveness of the antibody in treating cancer. For example, cysteine residue(s) can be introduced into the Fe region, thereby allowing interchain disulfide bond formation in this region. The homodimeric antibody thus generated can have improved internalization capability and/or increased complement-mediated cell killing and antibody-dependent cellular cytotoxicity (ADCC). See Caron, et al., J. Exp Med. 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al., Cancer Research 53:2560-2565 (1993). Alternatively, an antibody can be engineered that has dual Fc regions and can thereby have enhanced complement lysis and ADCC capabilities. See Stevenson, et al., Anti-Cancer Drug Design 3:219-230 (1989).

Methods for performing the uses listed above are well known to those skilled in the art. References disclosing such methods include without limitation “Molecular Cloning: A Laboratory Manual”, 2ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987.

Although the present invention has been described in terms of preferred embodiments, it is intended that the present invention encompass all modifications and variations that occur to those skilled in the art upon consideration of the disclosure herein, and in particular those embodiments that are within the broadest proper interpretation of the claims and their requirements.

All literature cited herein (scientific articles, U.S. patents, foreign patents, and published patent applications) is incorporated by reference.

What is claimed is:

1. A method activating CD45 phosphatase activity via administration of an anti-CD45 antibody capable of activating CD45 phosphatase activity.

2. The method of claim 1 wherein the antibody is a monoclonal antibody.

3. The method of claim 2 wherein the monoclonal antibody is a chimeric, humanized, or fully human antibody.

4. A method of inhibiting JAKs via administration of an anti-CD45 antibody capable of activating CD45 phosphatase activity.

5. The method of claim 4 wherein the antibody is a monoclonal antibody.

6. The method of claim 5 wherein the monoclonal antibody is a chimeric, humanized, or fully human antibody.

7. A method of controlling proliferation of hematopoietic cell lineages via administration of an anti-CD45 antibody capable of activating CD45 phosphatase activity.

8. The method of claim 7 wherein the antibody is a monoclonal antibody.

9. The method of claim 8 wherein the monoclonal antibody is a chimeric, humanized, or fully human antibody.

10. A method of controlling, decreasing, or preventing an inflammation process or disease via the administration of an anti-CD45 antibody capable of activating CD45 phosphatase activity.

11. The method of claim 10 wherein the antibody is a monoclonal antibody.

12. The method of claim 11 wherein the monoclonal antibody is a chimeric, humanized, or fully human antibody.

13. A method inhibiting CD45 phosphatase activity via administration of an anti-CD45 antibody capable of inhibiting CD45 phosphatase activity.

14. The method of claim 13 wherein the antibody is a monoclonal antibody.

15. The method of claim 14 wherein the monoclonal antibody is a chimeric, humanized, or fully human antibody.

16. A method of activation of JAKs via administration of an anti-CD45 antibody capable of inhibiting CD45 phosphatase activity.

17. The method of claim 16 wherein the antibody is a monoclonal antibody.

18. The method of claim 17 wherein the monoclonal antibody is a chimeric, humanized, or fully human antibody.

19. A method of increasing an immune response by suppressing CD45 activity via administration of an anti-CD45 antibody capable of inhibiting CD45 phosphatase activity.

20. The method of claim 19 wherein the antibody is a monoclonal antibody.

21. The method of claim 20 wherein the monoclonal antibody is a chimeric, humanized, or fully human antibody.
